(19) United States
(12) Patent Application Publication
(10) Pub. No.: US 2024/0150703 A1
(43) Pub. Date: May 9, 2024

(54) AUTOMATED ENUMERATION SYSTEM

(71) Applicant: Charles River Laboratories, Inc., Wilmington, MA (US)

(72) Inventors: Archibald DELORME, Wilmington, MA (US); Zichao BIAN, Wilmington, MA (US)

(21) Appl. No.: 18/487,284

(22) Filed: Oct. 16, 2023

Related U.S. Application Data
(60) Provisional application No. 63/382,128, filed on Nov. 3, 2022.
(60) Provisional application No. 63/380,665, filed on Oct. 24, 2022.

(51) Int. Cl. C12M 1/34; C12M 1/00; C12M 1/22; C12M 1/36; C12R 1/01; C12R 1/645; G06V 10/75; G06V 10/82; G06V 20/69

(52) U.S. Cl. CPC C12M 41/36; C12M 23/10; C12M 23/22; C12M 23/38; C12M 41/08; C12M 41/48; G06V 10/751; G06V 10/82; G06V 20/698; C12R 2001/01; C12R 2001/645

(57) ABSTRACT

The present disclosure provides systems and methods for detecting the presence of colonies on agar plates. The systems and methods are suitably useful for evaluating the presence of a contaminant (e.g., bacterial, fungal, etc.) in a sample, such as a raw material or component of a pharmaceutical composition, by analyzing whether the samples produce colonies on an agar plate. The systems utilize a lighting arrangement to increase the accuracy of the analysis, and in embodiments, include the use of artificial intelligence as part of an automated process.

### CROSS-REFERENCE

The present application claims the benefit of U.S. Provisional Patent Application Nos. 63/380,665, filed on Oct. 24, 2022, and 63/382,128, filed Nov. 3, 2022, both entitled “Automated Enumeration System” and naming as inventors Archibald Delorme and Zichao BIAN. The contents of each of these applications are incorporated herein by reference in their entireties.

### FIELD OF THE INVENTION

The present disclosure provides systems and methods for detecting the presence of and quantifying colonies on agar plates. The systems and methods are suitably useful for evaluating the presence of a contaminant (e.g., bacterial, fungal, etc.) in a sample, such as a raw material or component of a consumer or pharmaceutical composition, by analyzing whether the samples produce colonies on an agar plate. The systems utilize a lighting arrangement and a camera to increase the accuracy of the analysis, and in embodiments, include the use of artificial intelligence as part of an automated process.

### BACKGROUND OF THE INVENTION

Consumer and Pharmaceutical Quality Control (QC) departments often require a microbial detection and enumeration system to capture data from samples including raw materials, water (purified and water-for-injection), in-process materials, bulk drug substance, and environmental monitoring. Most QC relies on manual counting methods which are very subjective and time consuming. Emerging regulations question the accuracy of these methods and are urging the use of the 4-eyes concept, where one person counts the colonies on the plate and another person verifies the count for data integrity.

U.S. Pat. No. 9,290,382, provides an instrument for detecting microcolonies of target cells, but images the colonies through the lid of a petri dish, which can cause errors in detection.

What is needed is an automated system and method for colony counting that is highly reproducible and reduces the need for input from an operator. The present invention fulfills these needs.

### SUMMARY OF THE INVENTION

In embodiments, provided herein is a system for detecting a colony growing on an agar plate, the system comprising a photoelectric array detector having associated optics to detect a detection field of a surface of the agar plate, one or more illumination sources for illuminating the detection field, the illumination sources positioned to illuminate the agar plate, a collection lens system positioned between the photoelectric array detector and the agar plate, a computer programmed to receive data collected by the photoelectric array detector, wherein the data is a digital representation of the detection field, the colony growing on an agar within the plate is detected through a bottom surface of the agar plate and the agar, and the computer analyzes the data to quantify a number of colonies in the detection field.

In further embodiments, provided herein is a system for detecting a colony growing on an agar plate, the system comprising a photoelectric array detector having associated optics to detect a detection field of a back surface of the agar plate, a plurality of illumination sources encircling a side of the agar plate for illuminating the detection field, a collection lens system positioned between the photoelectric array detector and the agar plate, a computer programmed to receive data collected by the photoelectric array detector, wherein the data is a digital representation of the detection field, the colony growing on agar within the plate is detected through a bottom surface of the agar plate and the agar, and the computer analyzes the data to quantify a number of colonies in the detection field.

In additional embodiments, provided herein is a method for quantifying a number of colonies growing on an agar plate, method comprising illuminating a detection field of a back surface of the agar plate with one or more illumination sources, wherein the illumination sources are positioned to illuminate a surface of the agar plate, detecting the detection field with a photoelectric array detector having associated optics, the detecting occurring through a collection lens system positioned between the photoelectric array detector and the agar plate, receiving data collected by the photoelectric array detector as a digital representation of the detection field; and analyzing the data collected to quantify a number of colonies in the detection field.

Also provided herein is a method of training a neural network, comprising obtaining a test image of colonies growing on an agar plate, comprising illuminating a detection field of a surface of the agar plate with one or more illumination sources, wherein the illumination sources are positioned to illuminate the agar plate, detecting the detection field with a photoelectric array detector having associated optics, the detecting occurring through a collection lens system positioned between the photoelectric array detector and the agar plate, receiving data collected by the photoelectric array detector as a digital representation of the detection field, producing an image of cells in the detection field, providing a training set of images having a quantified number of colonies growing on the agar plate, comparing the test image to the training set of images via a computing system to generate an indication of the number of colonies in the test image, and repeating the obtaining, providing and comparing with a plurality of additional test images to train the neural network.

In additional embodiments, provided herein is a method for quantifying a number of colonies growing on an agar plate, method comprising illuminating a detection field of a surface of the agar plate with one or more illumination sources, wherein the illumination sources are positioned to illuminate a surface of the agar plate, detecting the detection field with a photoelectric array detector having associated optics, the detecting occurring through a collection lens system positioned between the photoelectric array detector and the agar plate, receiving data collected by the photoelectric array detector as a digital representation of the detection field, producing an image of colonies in the detection field, applying via a computer system, a trained neural network to the image of colonies to generate a quantitative determination of a number of colonies in the detection field, wherein the trained neural network has been trained with a training set of images which have been generated from images of the detection field where colonies growing on agar within the plate are detected through a surface of the agar plate and the agar, and displaying the quantitative determination of the number of colonies in the detection field.

### BRIEF DESCRIPTION OF THE DRAWINGS

FIGS. 1A-1B show exemplary agar plates.

FIG. 1C shows a side view of an agar plate.

FIG. 1D shows an exemplary system for detecting a colony growing on an agar plate, as described herein.

FIG. 2A shows a further exemplary system for detecting a colony growing on an agar plate, as described herein.

FIG. 2B shows an exemplary illumination source in accordance with embodiments described herein.

FIG. 3 shows a still further exemplary system for detecting a colony growing on an agar plate, as described herein.

FIG. 4 shows a system for detecting a colony growing on an agar plate, including a stage and robotic arm.

FIG. 5 shows a flow diagram illustrating an overall flow of methods and operations for the training of a neural network for automated colony detection implemented by a computer system.

FIG. 6 shows an exemplary agar plate used in a training set for the training of a neural network.

FIG. 7A show a computing system in accordance with embodiments herein.

FIG. 7B shows a non-transitory computer-readable medium in accordance with embodiments herein.

FIGS. 8A-8B show images of agar plates taken through the top cover.

FIGS. 9A-9B show images taken through the bottom and top, respectively, of an agar plate in accordance with embodiments herein.

FIGS. 10A-10B show images collected using systems described herein with the illumination source illuminating a side of the agar plate and the agar.

FIG. 11 shows an example of results with an algorithm applied using the images from the systems described herein.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps.

The systems, methods, processes and devices described herein are suitably useful for detecting, and ultimately providing a qualitative or quantitative assessment of colonies (e.g., bacterial, fungal, etc.) growing on an agar plate. As described herein, there is a need in consumer and pharmaceutical QC for such automated enumeration systems to capture data from samples including raw materials, water (purified and water-for-injection), in-process materials, bulk drug substance, and environmental monitoring. The ability to obtain an accurate, end-point count, using non-proprietary consumables (i.e., plates, media, or filters), is also desirable. However, the use of a traditional petri dish (also called agar plate herein) in such enumeration systems and methods can create a challenge when, to avoid contamination caused by mold forming spores and other contaminants, colonies are visualized through the lid of such plates, as condensation can often form, obscuring the ability to obtain a clear image for analysis by an automated system.

### Systems for Quantifying Colonies

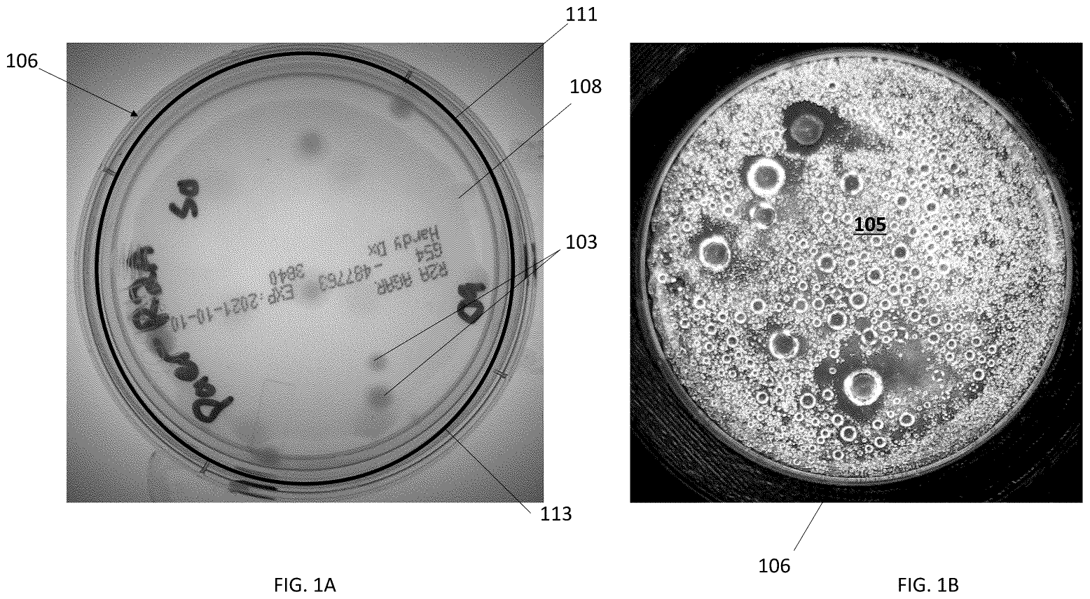

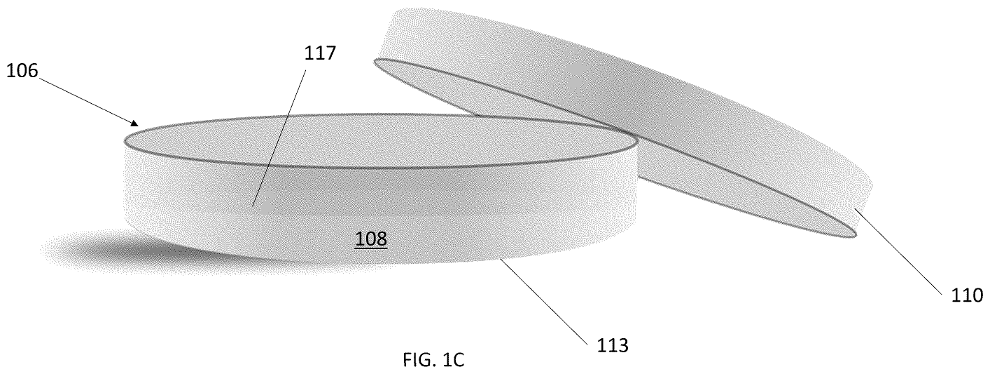

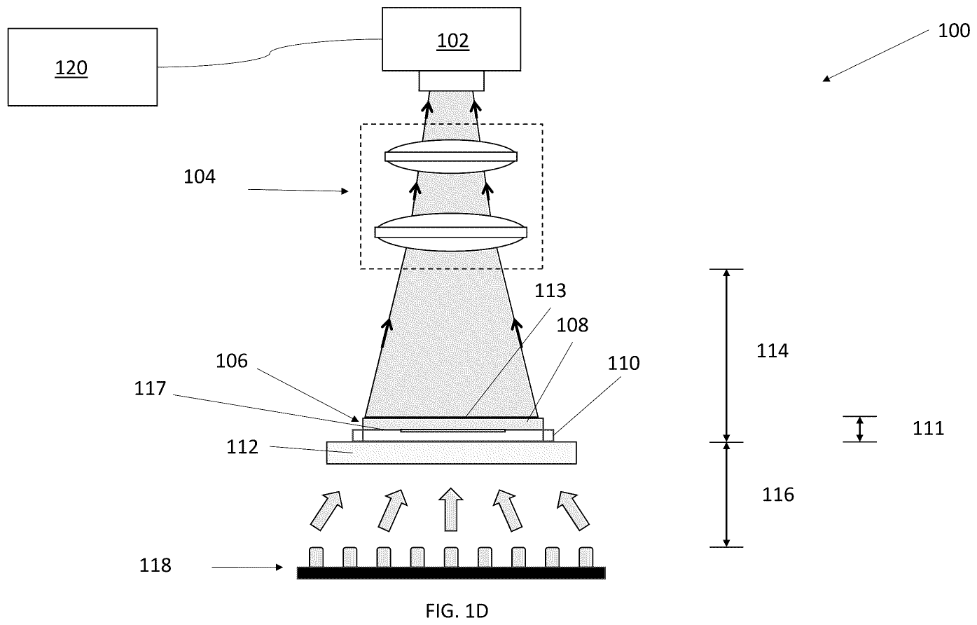

In embodiments, provided herein is a system for detecting a colony growing on an agar plate. FIG. 1A shows an agar plate 106 that includes multiple colonies 103 growing on agar 108 within the plate 106. Agar plates, also called petri dishes, culture plates, culture dishes, or plates, refer to shallow transparent lidded dishes, generally cylindrical in shape and comprised of plastic (though glass plates, including borosilicate glass, can also be used), that hold growth medium (agar) on which cells can be cultured. Suitably, the cells and the colonies are bacterial, fungal or yeast. As used herein a “colony” refers to a mass or group of cells of an organism (suitably bacterial, fungal or yeast) derived from the same mother cell, whether or not visible to the naked eye. A colony constitutes a clone of the mother cell that are all genetically alike. As shown in FIG. 1A, multiple individual colonies 103 are visible to the naked eye in an image taken of the agar plate. The colonies can grow an any suitable agar composition, including for example, galactose-based heterogenous polysaccharides. Agar can be composed of agarose and agaropectin polymers. A typical agar composition is 70% agarose and 30% agaropectin. Additional agar compositions are known in the art, and include for example, soy agar, Reasoner's 2A agar, blood agar, antibiotic treated agar, sabouraud agar, nutrient agar, etc. Colonies can also grow on an absorption pad or a membrane surface on the agar within the agar plate, including mixed cellulose ester (MCE) filter membranes, etc.

FIG. 1B shows another exemplary agar plate 106 on which condensation 105 has formed on the lid of the plate. During testing and analysis, it is desirable to keep the lid on the agar plate to avoid contamination. As is readily apparent, however, condensation obscures the ability to view any colonies 103 growing on agar within the plate, and thus it is not possible to obtain an accurate assessment of the number of colonies growing in the agar plate. The present systems, methods and devices allow for the detection and enumeration of a colony growing on an agar plate, even with the presence of condensation 105.

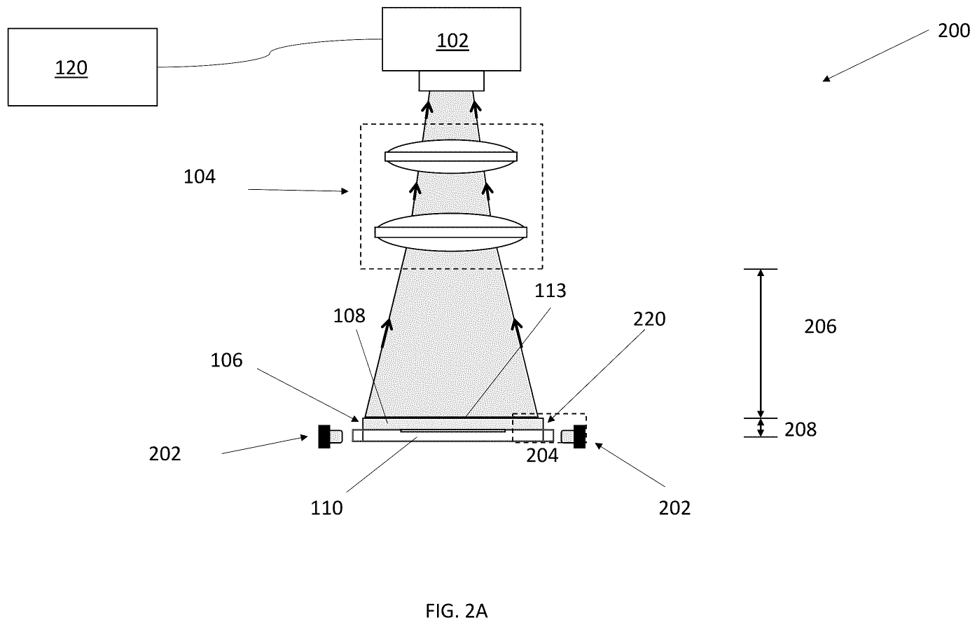

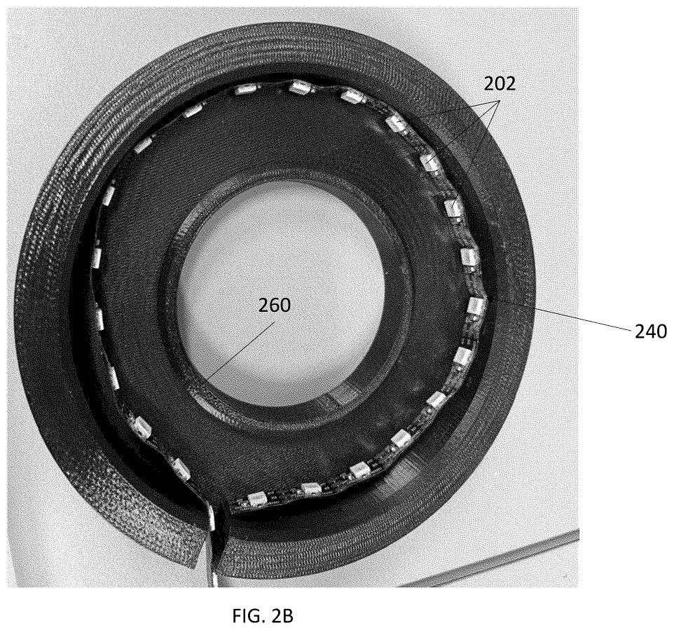

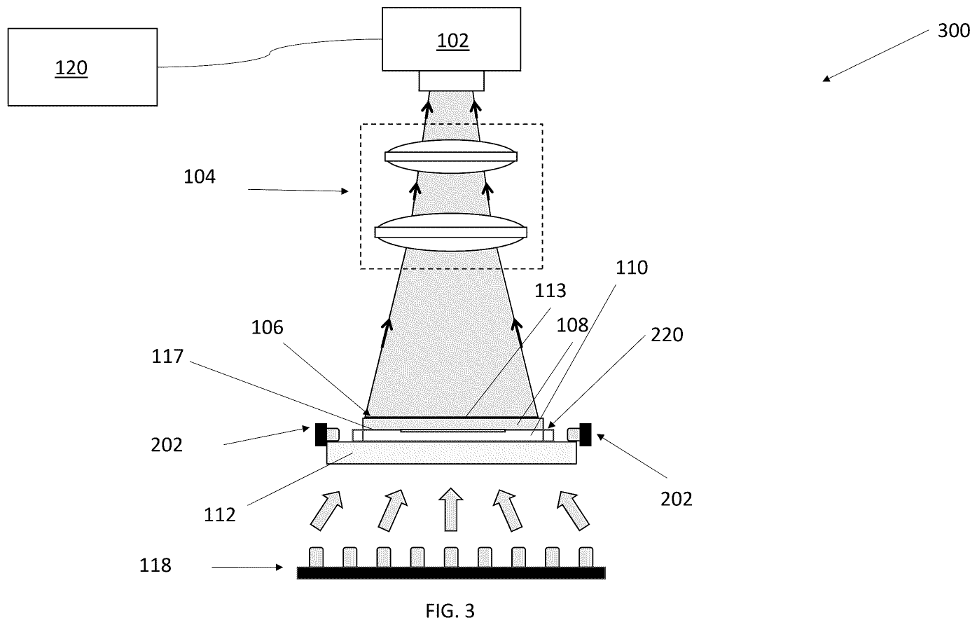

FIG. 1D, FIG. 2A and FIG. 3 show exemplary systems for detecting a colony growing on an agar plate, as described herein. In the systems described herein, colonies growing on the agar plate are detected through a surface of the agar plate, and suitably, are detected through a bottom surface 113 of the agar plate 106.

The system 100 shown in FIG. 1D suitably includes a photoelectric array detector 102 having associated optics to detect a detection field of the agar plate 106. FIG. 1A shows an exemplary detection field 111 of a bottom surface 113 of the agar plate 106. As shown, in embodiments, detection field 111 can suitably be the entire bottom surface 113 (i.e. the entire circular area denoted by thick, black line) of agar plate 106. In other embodiments, detection field 111 can be a section or portion of the entire bottom surface 113. In further embodiments, the detection field 111 can be any section or portion of agar 108. FIG. 1C shows a side view of an agar plate illustrating the bottom surface 113, agar 108, agar top surface 117, and top cover 110 (also called the lid) of the agar plate.

The systems for detecting a colony growing on an agar plate as described herein suitably include one or more illumination sources for illuminating the detection field 111. As described herein, the illumination sources are suitably positioned to illuminate the agar plate. As used herein an “illumination source” refers to a light, laser, combination of lighting sources, or other elements that provide light to allow data from the detection field to be collected by the photoelectric array detector.

In system 100, illumination source 118 illuminates agar top surface 117 of the agar plate 106 and the agar 108. As shown in FIG. 1D, illumination source 118 is suitably positioned such that light shines through the top surface 117 of the agar plate 106, through the agar 108 itself, illuminating the detection field 111, and out the bottom surface 113 of the agar plate.

Light then passes through a collection lens system 104 positioned between the photoelectric array detector 102 and the agar plate 106. System 100 also includes a computer 120 programmed to receive data collected by the photoelectric array detector 102. Suitably, the data is a digital representation of the detection field 111. A colony 103 growing on an agar 108 within the agar plate 106 (suitably on agar top surface 117) is detected through a surface (suitably the bottom surface 113) of the agar plate and the agar, and the computer analyzes the data to quantify a number of colonies 103 in the detection field 111. In embodiments, collection lens system 104 is a fixed focal length lens suitably having a large field of view (e.g., a lens having a working distance of about 100-200 mm, including a 16 mm fixed focal length lens). In other embodiments, the collection lens system 104 can be a telecentric lens, a microscopy objective lens, a liquid lens, a zoom-in lens, or other lens system that could collect light output from sample plate and focus it onto the detection surface of the photoelectric array detector. The distance between the photoelectric array detector and the detection field may vary and may be in the range of between 0-5 centimeters, 5-10 centimeters, 10-15 centimeters, 10-20 centimeters, 20-25 centimeters, 25-30 centimeters, or more depending on the settings and specifications of the detector.

In such embodiments, system 100 provides a device and method for detecting and enumerating colonies growing on the agar, by obtaining an image through the bottom of the agar plate 113. By imaging the colonies through the bottom of the agar plate 113, the imaging issues related to condensation on the agar plate lid obscuring the imaging are reduced or eliminated, allowing for an accurate determination of the number of colonies growing on the plate even while the lid of the plate remains on the plate.

In exemplary embodiments, system 100 further includes a light diffuser 112 positioned between illumination source 118 and the agar plate 106. Exemplary light diffusers are known in the art, and include for example, various plastic, glass, or other material elements that function as a semi-transmitting material placed between the light source and agar plate 106 to spread out, diffuse, or disperse the light as it passes through the material. This material does not block or cut light, but redirects light as it passes through for a diffused light spread on the agar plate.

System 200 shown in FIG. 2A, represents an embodiment where one of the illumination sources 202 used for illuminating the detection field, illuminates a side 220 of the agar plate 106 and the agar 108 as the side 220 forms a perimeter that encircles the side of the agar plate 113. Suitably, illumination source 202 comprises a plurality of illumination sources encircling the side 220 of the agar plate 106. As used herein, a plurality of illumination sources, refers to 2 or more, suitably 3 or more, 4 or more, 5 or more, 6 or more, 7 or more, 8 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, or between about 10-40, about 20-40, or about 20-30, illumination sources 202.

In embodiments, illumination source 202 is a string 240 of individual illumination sources 202 (see FIG. 2B). Suitably, the plurality of illumination sources encircles the side 220 of the agar plate 106 at a position between the top cover 110 of the agar plate 106 and the agar top surface 117 of the agar 108. In other embodiments, the plurality of illumination sources can encircle the side 220 of the agar plate 106 at a position between the bottom surface of the agar plate 113 and the agar top surface 117 of the agar plate 106. In other embodiments, the illumination sources can encircle the side 220 of the agar plate 106 at position up to the lid 110 of the agar plate 106. FIG. 2A shows a region 204 within which suitably the illumination sources are positioned, relative to agar plate 106, such that the agar plate is illuminated. In embodiments, the string 240 of illumination sources 202 can be connected together, as shown in FIG. 2B, encircling the side 220 of the agar plate 106. FIG. 2B also shows a support 260, onto which an agar plate 106 can be positioned such that the side 220 of the agar plate 106 is illuminated. In other embodiments, however, separate, individual illumination sources 202 can simply be arranged such that they encircle the side 220 of the agar plate 106. As used herein “encircle” includes instances where the illumination sources illuminate the entire side 220 (and thus circumference) of the agar plate 106 in a circular, or substantially circular pattern of sources, as well as partially illuminate the circumference of the agar plate 106.

System 300, shown in FIG. 3, shows an embodiment that includes two illumination sources for illuminating the detection field, including the first illumination source 202 positioned such that it is capable of illuminating the side 220 of the agar plate 106 and the agar 108, along with the second illumination source 118 illuminating the top 117 of the agar plate 106 and the agar 108. In an embodiment, the illumination source 202 can be positioned such that mirrors can be used to direct the light to the agar plate to illuminate the detection field.

Illumination sources for use in the various systems and devices described herein are suitably light-emitting diodes (LEDs), though in other embodiments, the illumination sources can comprise one or more lasers (e.g., a 532 nm Frequency-Doubled Diode Laser), or include a combination of light sources, or other elements that provide light to allow data from the detection field to be collected.

The photoelectric array detector 102 is a device or instrument that transduces photonic signals into electric signals, and in embodiments, comprises a charge-coupled device (CCD) detector, a photomultiplier tube detector, a complementary metal-oxide-semiconductor (CMOS) detector, or a photodiode detector. Suitably, the CMOS detector is a CMOS camera.

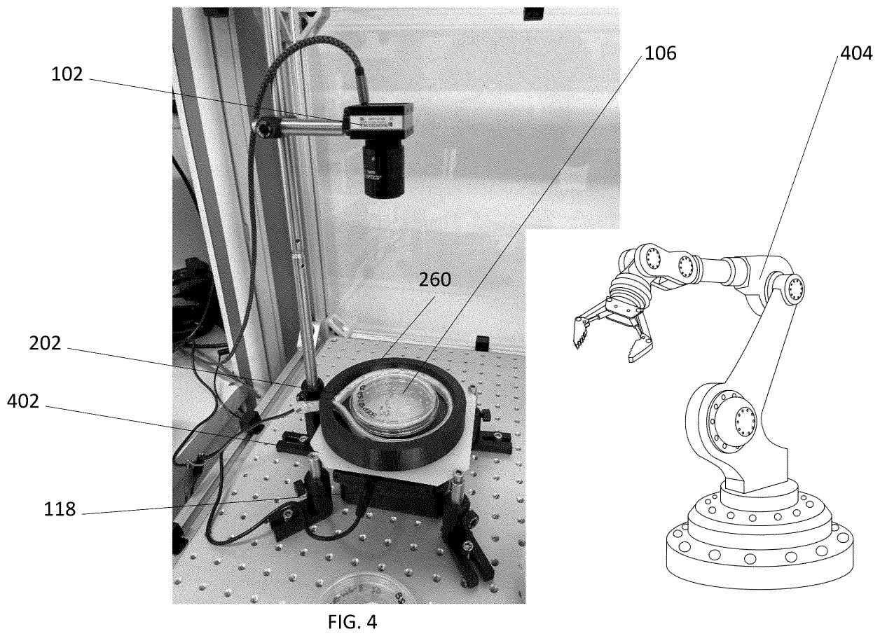

FIG. 4 shows a further embodiment in which a system described herein also includes a stage 402 for positioning the agar plate 106 relative to the illumination source 202 and/or 118 and the photoelectric array detector 102. In embodiments, the systems described herein can further include a robotic arm 404 for placing and removing the agar plate on the stage 402. Robotic arm 404 is show for illustrative purposes only and should not be taken as a limitation to the type of robotic arm that can be utilized in combination with the systems described herein.

As described throughout, it has been surprisingly found that the systems provided herein allow for the illumination and detection of colonies in the detection field, even when top cover 110 is present. While the top cover may contain condensation (e.g., from respiration of the colonies, or residual moisture in the agar), the systems and methods described herein allow for the detection of colonies growing on the agar, and reliable determination of the number of colonies.

In suitable embodiments, provided herein is a system for detecting colony 103 growing on agar plate 106, the system comprising photoelectric array detector 102 having associated optics to detect detection field 111 of back surface 113 of agar plate 106. Suitably, as described herein, the system includes a plurality of illumination sources 202 encircling side 220 of agar plate 106 for illuminating detection field 111. The system also suitably comprises collection lens system 104 positioned between photoelectric array detector 102 and agar plate 106. As described herein, the systems further include computer 120 programmed to receive data collected by photoelectric array detector 102. As described herein, the data is a digital representation of detection field 111, the colony 103 growing on agar 108 within the plate 106 is detected through the bottom surface 113 of the agar plate 106 and the agar 108, and the computer 120 analyzes the data to quantify a number of colonies 103 in the detection field 111.

As described herein, suitably the systems further include second illumination source 118 illuminating agar top surface 117 of the agar plate 106 and the agar 108, and suitably light diffuser 112 positioned between the second illumination source and the agar plate. Suitably, the plurality of illumination sources 202 encircle the side of the agar plate at a position between the bottom surface of the agar plate and the top of the agar plate, for example, in the form of a string 240 of lights (such as LED lights or laser light sources).

Exemplary photoelectric array detectors are described herein, including for example, a charge-coupled device (CCD) detector, a photomultiplier tube detector, a complementary metal-oxide-semiconductor (CMOS) detector (including a CMOS camera), or a photodiode detector.

In suitable embodiments, the systems can further include stage 402 for positioning the agar plate 106 relative to the illumination sources 202 and the photoelectric array detector 102, as well as robotic arm 404 for placing and removing the agar plate on the stage.

### Automated Methods for Quantifying Colonies

In further embodiments, provided herein is a method for quantifying a number of colonies growing on an agar plate. The methods suitably include illuminating a detection field on a back surface of the agar plate with one or more illumination sources, wherein the illumination sources are positioned to illuminate a surface of the agar plate.

The methods further include detecting the detection field with a photoelectric array detector having associated optics, the detecting occurring through a collection lens system positioned between the photoelectric array detector and the agar plate. Data collected by the photoelectric array detector is received as a digital representation of the detection field. The methods further include analyzing the data collected to quantify the number of colonies in the detection field.

Suitably, the methods are automated, in that input from a user is not required to position the agar plate, illuminate the plate, and/or collect and analyze the data, and instead these elements are performed in machine-driven process. This automation reduces the possibility for contamination of the agar plates with external bacteria, etc., and allows for a high-throughput method to analyze a large number of agar plates (and thus samples) quickly and easily.

As described herein, suitably two illumination sources are used for illuminating the detection field, wherein a first illumination source illuminates a side of the agar plate and the agar. Suitably the first illumination source comprises a plurality of illumination sources encircling the side of the agar plate, for example between the bottom surface of the agar plate and the top of the agar plate. In embodiments, a second illumination source illuminates a top of the agar plate and the agar, and suitably the second illumination source illuminates the agar plate through a light diffuser positioned between the second illumination source and the agar plate.

Exemplary illumination sources, lens systems and photoelectric array detectors are described herein.

### Methods of Training a Neural Network and Methods of Quantifying Using a Artificial Intelligence

In further embodiments, provided herein is a method of training a neural network, comprising obtaining a test image of colonies growing on an agar plate, the obtaining comprising: illuminating a detection field on a surface of the agar plate with one or more illumination sources, wherein the illumination sources are positioned to illuminate the agar plate; detecting the detection field with a photoelectric array detector having associated optics, the detecting occurring through a collection lens system positioned between the photoelectric array detector and the agar plate; receiving data collected by the photoelectric array detector as a digital representation of the detection field; producing an image of cells in the detection field, providing a training set of images having a quantified number of colonies growing on the agar plate; comparing the test image to the training set of images via a computing system to generate an indication of the number of colonies in the test image; and repeating the obtaining, providing and comparing with a plurality of additional test images to train the neural network.

Also provided herein is a method for quantifying a number of colonies growing on an agar plate, method comprising: illuminating a detection field on a surface of the agar plate with one or more illumination sources, wherein the illumination sources are positioned to illuminate a surface of the agar plate; detecting the detection field with a photoelectric array detector having associated optics, the detecting occurring through a collection lens system positioned between the photoelectric array detector and the agar plate; receiving data collected by the photoelectric array detector as a digital representation of the detection field; producing an image of colonies in the detection field, applying via a computer system, one or more trained neural networks, or AI algorithms, to the image of colonies to generate a quantitative determination of a number of colonies in the detection field, wherein the trained neural network has been trained with a training set of images which have been generated from images of the detection field where colonies growing on agar within the plate are detected through a surface of the agar plate and the agar; and displaying the quantitative determination of the number of colonies in the detection field.

One aspect of the embodiments herein relates to a computing system for processing end-point imaging of colonies using a trained neural network (or other form of machine learning) to count and analyze colonies, and to further improve the accuracy and precision of a colony counting system. The computing system (e.g., computer 120) may include at least one processing circuit, a non-transitory computer-readable medium, a display device, and a communication interface. The end-point images of the colonies may be derived from the photoelectric array detector having associated optics to detect the detection field. The end-point images may also represent a test image of colonies growing on an agar plate 106.

FIG. 5 provides a flow diagram illustrating an overall flow of methods and operations for the training of a neural network for automated colony detection implemented by a computer system. In an embodiment, the method 500 may include operations 502-514 which include receiving (or providing) a set of images 502, splitting the image set into two parts—one for training and one for validation 504, initializing the weights of the network 506, proceeding to a forward pass into the neural network 508, using a cost function for optimization of the parameters of the network 510, backward propagation with an optimization technique 512, and validating the training using the validation database 514, then repeating these elements with a plurality of additional images 508 to detect and quantify the number of colonies growing on a plurality of agar plates represented in the plurality of additional test images.

In an embodiment, an operation 502 includes receiving a set of images that include image data representing colonies 103 growing on an agar plate 106. The colonies 103 may include a range of bacteria, yeasts, and mold. The set of images may be generated by the systems as described herein. For example, generating the training set of images may include illuminating a detection field on a surface of the agar plate with one or more illumination sources, wherein the illumination sources are positioned to illuminate the agar plate. The detection field may be detected with a photoelectric array detector having associated optics. The detection may occur through a collection lens system positioned between the photoelectric array detector as a digital representation of the detection field. An image of colonies in the detection field may be produced as the generated data for use in the training set(s).

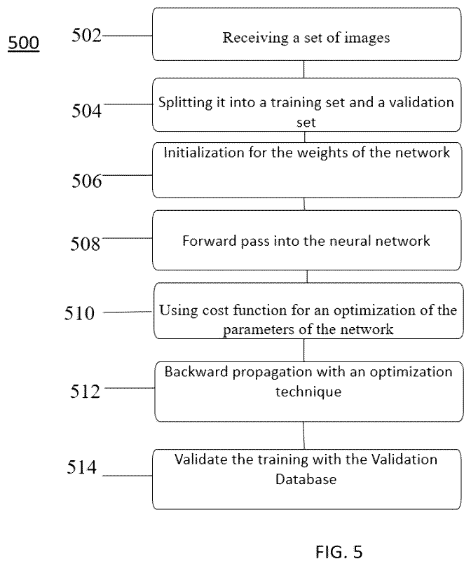

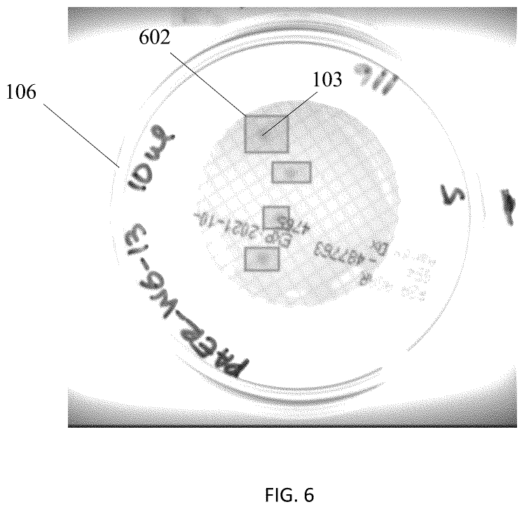

After receiving the set of images, the method 500 may include operation 504 where the computer system splits the set of images into two parts that maybe used for the training and the validation of the neural network to learn how to automatically detect and quantify the number of colonies growing on the agar plate. For example, the training set of images may include a first set of images that represent agar plates containing colonies 103 (i.e., bacteria, yeasts, and/or mold), and include a second set of images that represent agar plates with no colonies growing. In an embodiment, at least one image of the training set of images may include an agar plate containing a number of overlapping or merged colonies. An annotation may be associated with the images composing the set of images to indicate the presence of objects of interest on the image. For example an image composing the set of images used for training might have some objects on it that would be described by annotations. In an embodiment, annotations may indicate the number or count on each of the training set images or on each of the validation set of images. In an embodiment, the annotations may indicate or identify the type of microorganism(s) present. The annotations may be manually created or applied to each images in the set of images. For example, one image from the training set of images may include a plurality of growths, or colonies 103, present on the agar plate 106. A bounding box 602 may then be applied to each colony 103 present in the image, with an associated training score, as exemplified in FIG. 6.

The method 500 may include operation 506 where the weights of the network are initialized, allowing each nodes of the network to be associated with a specific value. For example, for the training of the algorithm the weights may be initialized with random weights or they may be initialized with weights from a network trained with similar kind of data.

The method 500 may include operation 508 where the computer system receives a training image having a quantified number of colonies growing on the agar plate. Generating the training image may include a number of steps. For example, generating the test image may include illuminating a detection field on a surface of the agar plate with one or more illumination sources, wherein the illumination sources are positioned to illuminate the agar plate. The detection field may be detected with a photoelectric array detector having associated optics. The detection may occur through a collection lens system positioned between the photoelectric array detector as a digital representation of the detection field. An image of colonies in the detection field may be produced as the generated data for use in the training set(s). In embodiments, some annotations may be added to the image to specify the object of interest existing in the image.

The method 500 may include operation 508 where an image that may be created as described in the previous paragraph is sent into the neural network (i.e., a forward pass into the neural network). The goal of this step may be to recover a feature map corresponding to the image and to extract the information of interest from this feature map. In an embodiment, the neural network may be an automated neural network, a convolutional neural network, a corner neural network, or any other type of neural network For example, the neural network may be a convolutional neural network having a sequence of convolutional layers, which may include a first convolutional layer configured to apply a convolutional filter on an input test image to generate an activation map, and further include additional convolutional layers configured to apply respective convolutional filters on respective activation maps or other outputs of previous convolutional filters in the sequence. The sequence of convolutional layers may each include a respective activation layer, which may apply an activation function (e.g., a rectified linear unit (ReLU) function) to an output of a convolutional filter in the convolutional layer. In an embodiment, the training of the neural network may include the computing system adjusting weights or other parameter values of the convolutional filters or other components of the convolutional layers, so as to cause the sequence of convolutional layers to convert the set of training images to output values equivalent to the annotations used for training, or at least present a low amount of deviation from the annotations used for training.

The method 500 may include operation 510 where a cost function is defined that may be used to describe how well the network is doing on each of its nodes. In an embodiment the cost function may be a regression cost function, a binary classification cost function or a Multi-class classification cost function. The implemented cost function applied to the neural network may be able to detect the how well the neural network fit the data.

The method 500 may include the operation 512 where the computer systems proceed to a backward propagation on the neural network. During this step the weights of the network may be tuned in order for the final predicted result to match the expectation.

The method 500 may include the operation 514 where the neural network is applied to the second part of the set of images, the validation dataset. From this step we may be able to verify that the network properly learned from the first part of the set of images used for the training of the neural network. This could be done to compare the annotation created by the network as a response to the set of validation images as an input and to compare them with the annotations created

Operations 506-508 may then be repeated and applied to a plurality of set images for automatically and efficiently identifying colonies via the trained neural network. In subjecting the computer system to this method, the detection/counting of colonies present on an agar plate may be achieved at a minimum 80% precision rate, more preferably at a 90% precision rate or a 95% precision rate, with a maximum recall to avoid any undetected colonies.

The above embodiments may be implemented separately, or may be combined. For example, the combination may involve the use of one or more neural networks to extract an image portion from the test image that represents a colony on the agar plate, to determine the level of growth of the colony on the agar plate, to determine the total number of colonies present on the agar plate, and to determine the presence of moisture/water within or on the agar plate. Such a combination may use a single neural network, or may involve multiple neural networks.

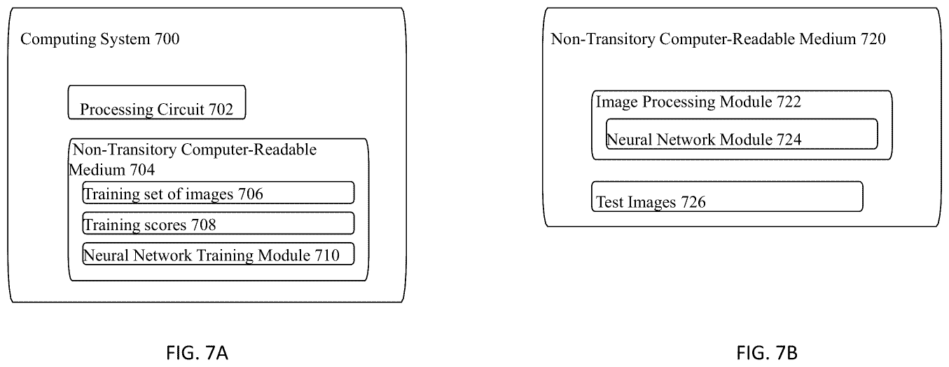

FIG. 7A depicts a computing system 700 that is configured to train a neural network to output the information as described above. In an embodiment, the computing system includes a processing circuit 702 configured to execute a neural network training module 710 configured to train a neural network based on the set of training images 706 and the outputted training scores 708 The neural network training module 710 may rely on machine learning platforms and frameworks, such as TensorFlow, PyTorch, Keras, OpenCV, and the like. In an embodiment, the neural network may be trained on a large set of training images 706 to reflect a broad range of colony growth values, size of colonies, instances of colony overlap, size of agar plates, presence of moisture, and types of microorganisms. FIG. 7B shows elements of non-transitory computer-readable medium 720, which can include image processing module 722, neural network module 724 and test images 726.

### EXAMPLES

### Construction of a System for Detecting Colonies on an Agar Plate

A system for detecting colonies on an agar plate as described herein was constructed.

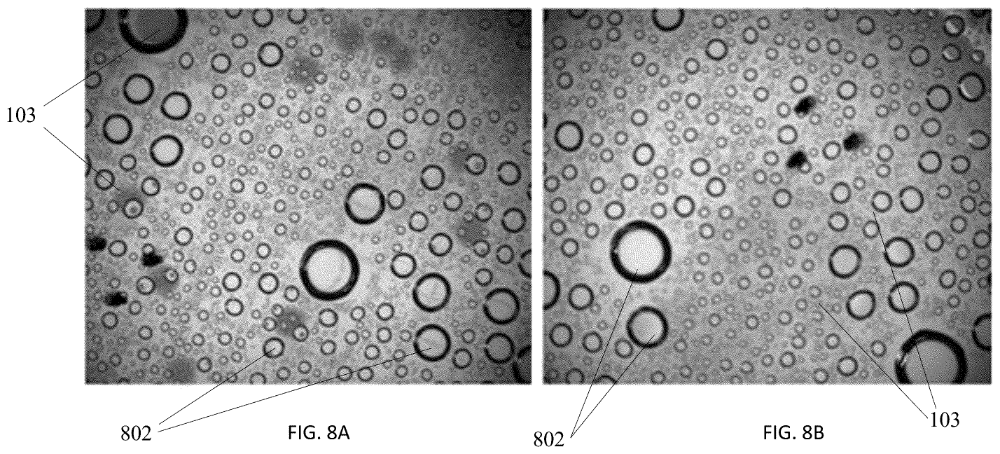

Initial testing was conducted by imaging agar plates through the top cover in place, in order to minimize contamination of the plate. The resulting images, however, were of poor quality due to condensation on the lid. See FIGS. 8A-8B. As noted, condensation droplets 802 obscure colonies 103 growing below. Use of an automated imaging and counting system to quantify these colonies was not successful, as many of the colonies were not counted, or condensation droplets were counted as colonies.

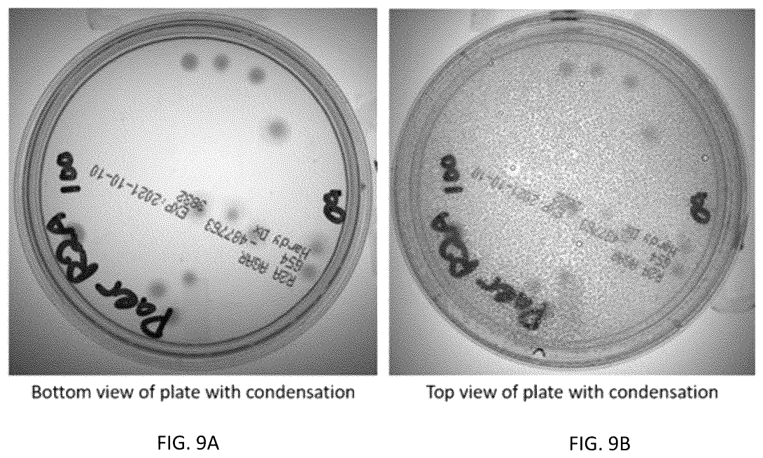

Instead of imaging through the plate from top cover, a system as described herein was designed to image the plate from the bottom (plate is flipped upside down). Suitably, a diffuser is placed close to the lid of the plate (ideally directly in contact with or very close to the agar plate lid), allowing evenly distributed illumination onto the sample but also aiding in eliminating impact due to condensation. A white diffuser, 3 mm in thickness with 45% transmission was selected, though other diffusers can also be utilized. A photoelectric array detector 104 (fixed focal length lens) was placed between about 100 mm to about 180 mm (suitably about 160 mm) from the agar plate 106 (see 114 in FIG. 1D). The illumination source 118 was placed between about 15 mm to about 20 mm (suitably about 16 mm) below the agar plate 106 (see 116 in FIG. 1D). Images collected utilizing this system through the bottom of the agar plate significantly reduced the obscuring effect of condensation (see FIG. 9A) relative to images take through the top of the plate, with condensation (see FIG. 9B).

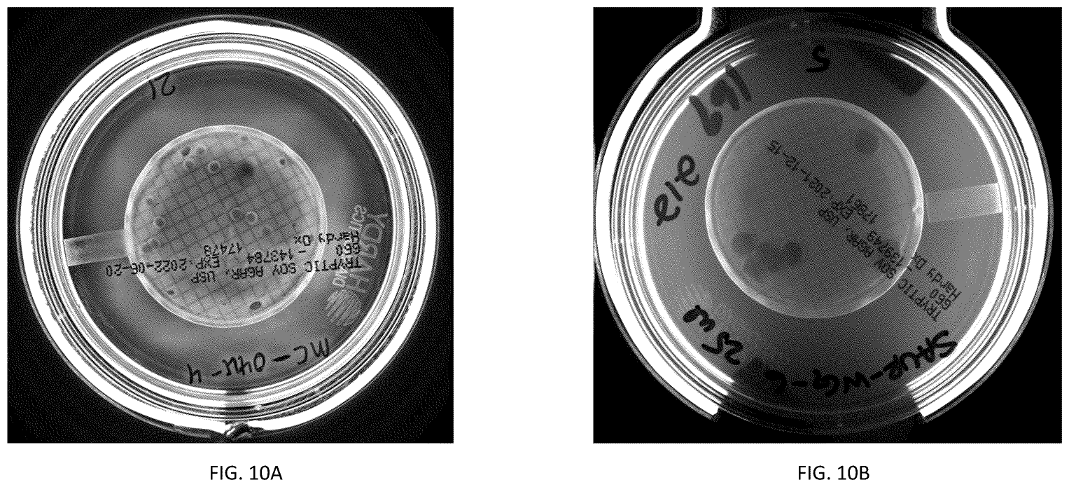

An additional embodiment was designed where an illumination source illuminates a side of the agar plate and the agar. A plurality of illumination sources was designed to encircle the side 220 of the agar plate at a position between the top cover 110 of the agar plate 106 and the agar surface 117 of the agar plate 106. Suitably, the plurality of illumination sources was in the form of a ring 240 of LED lights. A photoelectric array detector 104 (fixed focal length lens) was placed between about 100 mm to about 180 mm (suitably about 160 mm) from the agar plate 106 (see 206 in FIG. 2A). Suitably, the illumination sources are positioned so that the angle of the light source relative to horizontal plane of the lid of the agar plate is within about 0°, 1°, 2°, 3°, 4°, 5°, 6° or 7° or even 10°, and within about 3 mm in vertical orientation relative to the top of the agar plate, or 9 mm to the bottom of the agar plate (see 208 in FIG. 2A). In some embodiments, the angle of the light relative to the horizontal plane of the lid is 2° or 5°. FIGS. 10A-10B show various images collected using the system described herein with the illumination source illuminating a side of the agar plate and the agar. As can be seen, colonies growing on a membrane within the agar plate can also be clearly seen using this system design.

To further enhance the image contrast for better colony identification, a Contrast Limited Adaptive Histogram Equalization (CLAHE) algorithm is applied to the images.

### Feasibility Study and Machine Learning Approach

To support an initial feasibility study, microorganism culture plates were prepared for imaging. The focus was on water bioburden samples which includes primarily gram-negative bacteria filtered onto 0.45 μm white gridded (MCE) filter membranes, plated on R2A media, and incubated for 3-5 days.

5 organisms were chosen for this initial feasibility study:
Burkholderia cepacia ATCC 25416
Pseudomonas aeruginosa ATCC 9027
Bacillus subtilis ATCC 6633
Aspergillus brasiliensis ATCC 16404
Staphylococcus aureus ATCC 6538

Media plates included Tryptic Soy Agar (TSA), Reasoner's 2A (R2A), and Sabouraud Dextrose Agar (SDA). Filter membranes included Microfunnel Filter Funnels with Supor (MCE) 0.45 μm white gridded membranes, Microfunnel Filter Funnels with Supor 0.2 μm white gridded and plain white (non-gridded) filter membranes.

All stock microorganisms were from EZ-Accu Shot™. Lyophilized cultures (Microbiologics) and were prepared per manufacturer's instructions. Based on the label claim colony forming unit (CFU) count per microorganism, the volume added to each sample varied to give a range of colony growth between 0-50 CFU per PRD requirements. Negative control plates with filter membranes were prepared with sterile water only for baseline calculations for the machine learning algorithms.

All samples were incubated at 30° C. and plates were reviewed after 48-hours, 72-hours and at Day 5 of incubation. Multiple days were imaged for creation of a larger sample set for machine learning assessment. This gave the opportunity to capture images of microorganisms at various colony sizes and morphologies. A total of 213 images were taken using the system described herein. The samples contained varying amounts of condensation to no condensation.

Once the images were collected, Make Sense AI (makesense.ai) was used to annotate each image, so that the data created for each image can be inputted for machine learning development.

The images were imported and bounding boxes 602 were created manually around each colony 103 (area of interest). See FIG. 6. The labels for each microorganism tested were created as an additional step to enhance the detection algorithms. Once the annotations are completed, the data were exported as XML files.

The manual counting of colonies from the images demonstrated the ability to accurately count colonies using the system described herein, when compared to the compendial (standard) method.

TABLE 1
Comparison of Manual Counting using Imaging System

| | Compendial | Manual Count of |
|---|---|---|
| Microorganism | count | Colonies from Image |
| Burkhoderia cepacia | 8 | 8 |
| Psuedomonas aeruginosa | 4 | 4 |
| Bacillus subtilis | 10 | 10 |
| Aspergillus brasiliensis | 10 | 11 (small colony visible |
| | | but difficult to see by |
| | | the human eye) |
| Staphylococcus aureus | 8 | 8 |

### Machine Learning Concept and Summary of Initial Results

A machine learning algorithm was developed to detect/count microorganism colonies present on the agar plate by achieving a minimum precision of 95% with a maximum recall to avoid any undetected bacterial colonies.

### Hardware and Algorithms Used

The computer used to process the algorithm was an Intel i7-10700 CPU with 32 GB of RAM and equipped with an RTX 3070 Nvidia GPU with 8 GB of memory.

A generic artificial intelligence architecture was selected. It is an instance segmentation architecture adapted to be usable on custom datasets.

### Results of Machine Learning Algorithm Development

Database taken with System Described Herein

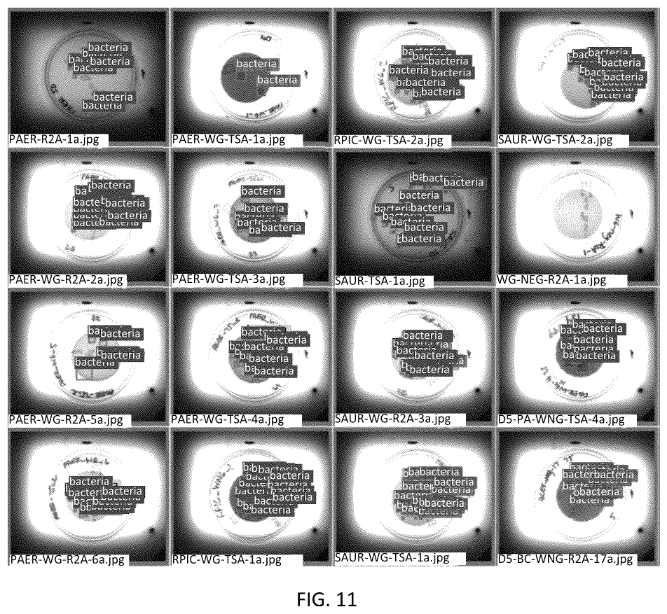

FIG. 11 shows an example of the results with the algorithm applied using the Images from the system described herein. Table 2 below shows the results of the algorithm application.

TABLE 2
Inverted Imaging System

| Precision | Recall | Database | mAp: 0.5 | mAP [0.5: 0.95] | Pretrained |
|---|---|---|---|---|---|
| 0.96 | 0.945 | AES Prototype | 0.963 | 0.51 | Yes |

| | |
|---|---|
| Explanation of | The results demonstrated that the images are providing more exploitable |
| the results | information than the previous databases. The precision and recall are |
| | higher and closer to the requirements. This may be due to the high |
| | contrast reached in the images. Since the filter membrane is illuminated |
| | with a strong light source the colonies are clearly distinguishable in the |
| | images allowing to reach these high levels. The results obtained will |
| | increase once a larger database of images is obtained. |

The results obtained with the system described herein support the feasibility and demonstrate a high image quality and contrast achieved with the imaging system.

### Embodiments

Embodiment 1 is a system for detecting a colony growing on an agar plate, the system comprising: a photoelectric array detector having associated optics to detect a detection field of a surface of the agar plate; one or more illumination sources for illuminating the detection field, the illumination sources positioned to illuminate the agar plate; a collection lens system positioned between the photoelectric array detector and the agar plate; a computer programmed to receive data collected by the photoelectric array detector, wherein the data is a digital representation of the detection field, the colony growing on an agar within the plate is detected through a bottom surface of the agar plate and the agar, and the computer analyzes the data to quantify a number of colonies in the detection field.

Embodiment 2 includes the system of Embodiment 1, wherein two illumination sources are used for illuminating the detection field, and wherein a first illumination source illuminates a side of the agar plate and the agar.

Embodiment 3 includes the system of Embodiment 2, wherein a second illumination source illuminates a top surface of the agar.

Embodiment 4 includes the system of Embodiment 3, further comprising a light diffuser positioned between the second illumination source and the agar plate.

Embodiment 5 includes the system of any of Embodiments 1-4, wherein the first illumination source comprises a plurality of illumination sources encircling the side of the agar plate.

Embodiment 6 includes the system of Embodiment 5, wherein the plurality of illumination sources encircles the side of the agar plate at a position between the top cover of the agar plate and the top of the agar.

Embodiment 7 includes the system of any of Embodiments 1-6 wherein the colony is growing on an absorption pad or a membrane surface on the agar within the agar plate.

Embodiment 8 includes the system of any of Embodiments 1-7, wherein the illumination sources comprise one or more lasers.

Embodiment 9 includes the system of any of Embodiments 1-7, wherein the illumination sources comprise one or more light-emitting diodes.

Embodiment 10 includes the system of any of Embodiments 1-9, wherein the photoelectric array detector comprises a charge-coupled device (CCD) detector, a photomultiplier tube detector, a complementary metal-oxide-semiconductor (CMOS) detector, or a photodiode detector.

Embodiment 11 includes the system of Embodiment 10, wherein the CMOS detector is a CMOS camera.

Embodiment 12 includes the system of any of Embodiments 1-13, further comprising a stage for positioning the agar plate relative to the illumination source and the photoelectric array detector.

Embodiment 13 includes the system of Embodiment 12, further comprising a robotic arm for placing and removing the agar plate on the stage.

Embodiment 14 includes the system of any of Embodiments 1-13, wherein the colony is a colony of bacteria cells or a colony of fungal cells.

Embodiment 15 includes the system of any of Embodiments 1-14, wherein a top cover of the agar plate is present during the illuminating and detecting.

Embodiment 16 includes the system of any of Embodiments 1-15, wherein the detection field is a bottom surface of the agar plate.

Embodiment 17 is a system for detecting a colony growing on an agar plate, the system comprising: a photoelectric array detector having associated optics to detect a detection field of a back surface of the agar plate; a plurality of illumination sources encircling a side of the agar plate for illuminating the detection field; a collection lens system positioned between the photoelectric array detector and the agar plate; a computer programmed to receive data collected by the photoelectric array detector, wherein the data is a digital representation of the detection field, the colony growing on agar within the plate is detected through a bottom surface of the agar plate and the agar, and the computer analyzes the data to quantify a number of colonies in the detection field.

Embodiment 18 includes the system of Embodiment 17, further comprising a second illumination source illuminates a top of the agar plate and the agar.

Embodiment 19 includes the system of Embodiment 18, further comprising a light diffuser positioned between the second illumination source and the agar plate.

Embodiment 20 includes the system of any of Embodiments 17-19, wherein the plurality of illumination sources encircle the side of the agar plate at a position between the top cover of the agar plate and the top of the agar.

Embodiment 21 includes the system of any of Embodiments 17-20, wherein the colony is growing on an absorption pad or a membrane surface on the agar within the agar plate.

Embodiment 22 includes the system of any of Embodiments 17-21, wherein the illumination sources comprise one or more lasers.

Embodiment 23 includes the system of any of Embodiments 17-21, wherein the illumination sources comprise one or more light-emitting diodes.

Embodiment 24 includes the system of any of Embodiments 17-23, wherein said photoelectric array detector comprises a charge-coupled device (CCD) detector, a photomultiplier tube detector, a complementary metal-oxide-semiconductor (CMOS) detector, or a photodiode detector.

Embodiment 25 includes the system of Embodiment 24, wherein the CMOS detector is a CMOS camera.

Embodiment 26 includes the system of any of Embodiments 17-25, further comprising a stage for positioning the agar plate relative to the illumination sources and the photoelectric array detector.

Embodiment 27 includes the system of Embodiment 26, further comprising a robotic arm for placing and removing the agar plate on the stage.

Embodiment 28 includes the system of any of Embodiments 17-27, wherein the colony is a colony of bacteria cells or a colony of fungal cells.

Embodiment 29 includes the system of any of Embodiments 17-28, wherein a top cover of the agar plate is present during the illuminating and detecting.

Embodiment 30 includes the system of any of Embodiments 17-29, wherein the detection field is a back surface of the agar plate.

Embodiment 31 is a method for quantifying a number of colonies growing on an agar plate, method comprising: illuminating a detection field of a back surface of the agar plate with one or more illumination sources, wherein the illumination sources are positioned to illuminate a surface of the agar plate; detecting the detection field with a photoelectric array detector having associated optics, the detecting occurring through a collection lens system positioned between the photoelectric array detector and the agar plate; receiving data collected by the photoelectric array detector as a digital representation of the detection field; and analyzing the data collected to quantify a number of colonies in the detection field.

Embodiment 32 includes the method of Embodiment 31, wherein two illumination sources are used for illuminating the detection field, and wherein a first illumination source illuminates a side of the agar plate and the agar.

Embodiment 33 includes the method of Embodiment 32, wherein a second illumination source illuminates a top of the agar plate and the agar.

Embodiment 34 includes the method any of Embodiments 31-33, further comprising a light diffuser positioned between the second illumination source and the agar plate.

Embodiment 35 includes the method any of Embodiments 31-34, wherein the first illumination source comprises a plurality of illumination sources encircling the side of the agar plate.

Embodiment 36 includes the method of Embodiment 35, wherein the plurality of illumination sources encircles the side of the agar plate at a position between the top cover of the agar plate and the top of the agar.

Embodiment 37 includes the method any of Embodiments 31-36, wherein the colony is growing on an absorption pad or a membrane surface on the agar within the agar plate.

Embodiment 38 includes the method any of Embodiments 31-37, wherein the illumination sources comprise one or more lasers.

Embodiment 39 includes the method any of Embodiments 31-37, wherein the illumination sources comprise one or more light-emitting diodes.

Embodiment 40 includes the method any of Embodiments 31-39, wherein said photoelectric array detector comprises a charge-coupled device (CCD) detector, a photomultiplier tube detector, a complementary metal-oxide-semiconductor (CMOS) detector, or a photodiode detector.

Embodiment 41 includes the method of Embodiment 40, wherein the CMOS detector is a CMOS camera.

Embodiment 42 includes the method any of Embodiments 31-41, further comprising positioning the agar plate on a stage relative to the illumination source and the photoelectric array detector.

Embodiment 43 includes the method of Embodiment 42, further comprising placing and removing the agar plate on the stage via a robotic arm.

Embodiment 44 includes the method any of Embodiments 31-43, wherein the colony is a colony of bacteria cells or a colony of fungal cells.

Embodiment 45 includes the method any of Embodiments 31-44, wherein a top cover of the agar plate is present during the illuminating and detecting.

Embodiment 46 includes the method any of Embodiments 34-45, wherein the detection field is a back surface of the agar plate.

Embodiment 47 is a method of training a neural network, comprising: obtaining a test image of colonies growing on an agar plate, comprising: illuminating a detection field of a surface of the agar plate with one or more illumination sources, wherein the illumination sources are positioned to illuminate the agar plate; detecting the detection field with a photoelectric array detector having associated optics, the detecting occurring through a collection lens system positioned between the photoelectric array detector and the agar plate; receiving data collected by the photoelectric array detector as a digital representation of the detection field; producing an image of cells in the detection field, providing a training set of images having a quantified number of colonies growing on the agar plate; comparing the test image to the training set of images via a computing system to generate an indication of the number of colonies in the test image; and repeating the obtaining, providing and comparing with a plurality of additional test images to train the neural network.

Embodiment 48 includes the method of Embodiment 47, wherein the neural network is a convolutional neural network or a corner neural network.

Embodiment 49 is a method for quantifying a number of colonies growing on an agar plate, method comprising: illuminating a detection field of a surface of the agar plate with one or more illumination sources, wherein the illumination sources are positioned to illuminate a surface of the agar plate; detecting the detection field with a photoelectric array detector having associated optics, the detecting occurring through a collection lens system positioned between the photoelectric array detector and the agar plate; receiving data collected by the photoelectric array detector as a digital representation of the detection field; producing an image of colonies in the detection field, applying via a computer system, a trained neural network to the image of colonies to generate a quantitative determination of a number of colonies in the detection field, wherein the trained neural network has been trained with a training set of images which have been generated from images of the detection field where colonies growing on agar within the plate are detected through a surface of the agar plate and the agar; and displaying the quantitative determination of the number of colonies in the detection field.

Embodiment 50 includes the method of Embodiment 49, wherein the neural network is a convolutional neural network or a corner neural network.

Embodiment 51 includes the method of Embodiment 49 or Embodiment 50, wherein two illumination sources are used for illuminating the detection field, and wherein a first illumination source illuminates a side of the agar plate and the agar.

Embodiment 52 includes the method of Embodiment 51, wherein a second illumination source illuminates a top of the agar plate and the agar.

Embodiment 53 includes the method of Embodiment 52, further comprising a light diffuser positioned between the second illumination source and the agar plate.

Embodiment 54 includes the method any of Embodiments 49-53, wherein the first illumination source comprises a plurality of illumination sources encircling the side of the agar plate.

Embodiment 55 includes the method of Embodiment 54, wherein the plurality of illumination sources encircles the side of the agar plate at a position between the top cover of the agar plate and the top of the agar.

Embodiment 56 includes the method any of Embodiments 49-55, wherein the colony is growing on an absorption pad or a membrane surface on the agar within the agar plate.

Embodiment 57 includes the method any of Embodiments 49-56, wherein the illumination sources comprise one or more lasers.

Embodiment 58 includes the method any of Embodiments 49-56, wherein the illumination sources comprise one or more light-emitting diodes.

Embodiment 59 includes the method any of Embodiments 49-58, wherein said photoelectric array detector comprises a charge-coupled device (CCD) detector, a photomultiplier tube detector, a complementary metal-oxide-semiconductor (CMOS) detector, or a photodiode detector.

Embodiment 60 includes the method of Embodiment 59, wherein the CMOS detector is a CMOS camera.

Embodiment 61 includes the method any of Embodiments 49-60, further comprising positioning the agar plate on a stage relative to the illumination source and the photoelectric array detector.

Embodiment 62 includes the method of Embodiment 61, further comprising placing and removing the agar plate on the stage via a robotic arm.

Embodiment 63 includes the method any of Embodiments 49-62, wherein the colony is a colony of bacteria cells or a colony of fungal cells.

Embodiment 64 includes the method any of Embodiments 49-63, wherein a top cover of the agar plate is present during the illuminating and detecting.

Embodiment 65 includes the method any of Embodiments 49-64, wherein the detection field is a back surface of the agar plate.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

1. A system for detecting a colony growing on an agar plate, the system comprising:
(a) a photoelectric array detector having associated optics to detect a detection field of a surface of the agar plate;
(b) one or more illumination sources for illuminating the detection field, the illumination sources positioned to illuminate the agar plate;
(c) a collection lens system positioned between the photoelectric array detector and the agar plate;
(d) a computer programmed to receive data collected by the photoelectric array detector,
wherein the data is a digital representation of the detection field, the colony growing on an agar within the plate is detected through a bottom surface of the agar plate and the agar, and the computer analyzes the data to quantify a number of colonies in the detection field.

2. The system of claim 1, wherein two illumination sources are used for illuminating the detection field, and wherein a first illumination source illuminates a side of the agar plate and the agar, optionally wherein a second illumination source illuminates a top surface of the agar.

3. The system of claim 2, wherein a second illumination source illuminates a top surface of the agar.

4. The system of claim 3, further comprising a light diffuser positioned between the second illumination source and the agar plate.

5. The system of claim 2, wherein the first illumination source comprises a plurality of illumination sources encircling the side of the agar plate.

6. The system of claim 5, wherein the plurality of illumination sources encircles the side of the agar plate at a position between the top cover of the agar plate and the top of the agar.

7. The system of claim 1 wherein the colony is growing on an absorption pad or a membrane surface on the agar within the agar plate.

8. The system of claim 1, wherein the illumination sources comprise one or more lasers.

9. The system of claim 1, wherein the illumination sources comprise one or more light-emitting diodes.

10. The system of claim 1, wherein the photoelectric array detector comprises a charge-coupled device (CCD) detector, a photomultiplier tube detector, a complementary metal-oxide-semiconductor (CMOS) detector, or a photodiode detector.

11. The system of claim 10, wherein the CMOS detector is a CMOS camera.

12. The system of claim 1, further comprising a stage for positioning the agar plate relative to the illumination source and the photoelectric array detector and optionally a robotic arm for placing and removing the agar plate on the stage.

13. The system of claim 12, further comprising a robotic arm for placing and removing the agar plate on the stage.

14. The system of claim 1, wherein the colony is a colony of bacteria cells or a colony of fungal cells.

15. The system of claim 1, wherein a top cover of the agar plate is present during the illuminating and detecting.

16. The system of claim 1, wherein the detection field is a bottom surface of the agar plate.

17. A system for detecting a colony growing on an agar plate, the system comprising:
(a) a photoelectric array detector having associated optics to detect a detection field of a back surface of the agar plate;
(b) a plurality of illumination sources encircling a side of the agar plate for illuminating the detection field;
(c) a collection lens system positioned between the photoelectric array detector and the agar plate;
(d) a computer programmed to receive data collected by the photoelectric array detector,
wherein the data is a digital representation of the detection field, the colony growing on agar within the plate is detected through a bottom surface of the agar plate and the agar, and the computer analyzes the data to quantify a number of colonies in the detection field.

18-30. (canceled)

31. A method for quantifying a number of colonies growing on an agar plate, method comprising:
(a) illuminating a detection field of a back surface of the agar plate with one or more illumination sources, wherein the illumination sources are positioned to illuminate a surface of the agar plate;
(b) detecting the detection field with a photoelectric array detector having associated optics, the detecting occurring through a collection lens system positioned between the photoelectric array detector and the agar plate;
(c) receiving data collected by the photoelectric array detector as a digital representation of the detection field; and
(d) analyzing the data collected to quantify a number of colonies in the detection field.

32-46. (canceled)

47. A method of training a neural network, comprising:
(a) obtaining a test image of colonies growing on an agar plate, comprising:
i. illuminating a detection field of a surface of the agar plate with one or more illumination sources, wherein the illumination sources are positioned to illuminate the agar plate;
ii. detecting the detection field with a photoelectric array detector having associated optics, the detecting occurring through a collection lens system positioned between the photoelectric array detector and the agar plate;
iii. receiving data collected by the photoelectric array detector as a digital representation of the detection field;
iv. producing an image of cells in the detection field,
(b) providing a training set of images having a quantified number of colonies growing on the agar plate;
(c) comparing the test image to the training set of images via a computing system to generate an indication of the number of colonies in the test image; and
(d) repeating the obtaining, providing and comparing with a plurality of additional test images to train the neural network.

48. (canceled)

49. A method for quantifying a number of colonies growing on an agar plate, method comprising:
(a) illuminating a detection field of a surface of the agar plate with one or more illumination sources, wherein the illumination sources are positioned to illuminate a surface of the agar plate;
(b) detecting the detection field with a photoelectric array detector having associated optics, the detecting occurring through a collection lens system positioned between the photoelectric array detector and the agar plate;
(c) receiving data collected by the photoelectric array detector as a digital representation of the detection field;
(d) producing an image of colonies in the detection field,
(e) applying via a computer system, a trained neural network to the image of colonies to generate a quantitative determination of a number of colonies in the detection field, wherein the trained neural network has been trained with a training set of images which have been generated from images of the detection field where colonies growing on agar within the plate are detected through a surface of the agar plate and the agar; and
(f) displaying the quantitative determination of the number of colonies in the detection field.

50-65. (canceled)
